(19) United States
(12) Patent Application Publication
(10) Pub. No.: US 2006/0210481 A1
(43) Pub. Date: Sep. 21, 2006

(54) Medicament dispenser

(76) Inventors: Ignatius Loy Britto, Evreux (FR); Christophe Laroche, Evreux (FR); Verna Charlene Lo Clark, Durham, NC (US); Isabelle Denise Peyron, Evreux (FR)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/552,983

(22) PCT Filed: Apr. 20, 2004
(86) PCT No.: PCT/EP04/04247

§ 371 Date: Oct. 12, 2005

Related U.S. Application Data
(60) Provisional application No. 60/464,477, filed on Apr. 22, 2003.

(51) Int. Cl. A61L 9/04; A61M 11/00

(52) U.S. Cl. 424/45; 128/200.23

(57) ABSTRACT

A component of a medicament dispenser has a surface which, in use of the dispenser, contacts a medicinal formulation contained in the dispenser, said surface being presented by a fluorinated polymeric composition having CF₃CHFCF₃ as a monomer thereof.

### FIELD OF THE INVENTION

The present invention relates to a medicament dispenser and is particularly, but not exclusively, concerned with a pressurised metered dose inhaler (pMDI).

### BACKGROUND OF THE INVENTION

Drugs for treating respiratory and nasal disorders are frequently administered in aerosol formulations through the mouth or nose. One widely used method for dispensing such aerosol drug formulations involves formulating the drug as a suspension or a solution in a liquefied gas propellant. The suspension/solution is stored in a sealed canister capable of withstanding the pressure required to maintain the propellant as a liquid. The suspension/solution is dispersed by activation of a dose-metering valve affixed to the canister.

A metering valve generally comprises a metering chamber, which is of a set volume and is designed to administer per actuation an accurate predetermined dose of medicament. As the suspension/solution is forced from the metering chamber through the valve stem by the high vapour pressure of the liquid propellant, the propellant rapidly vaporises leaving a fast moving cloud of very fine particles of the drug formulation. This cloud of particles is directed into the nose or mouth of the patient by a channelling device such as a cylinder or open-ended cone. Concurrently with the activation of the aerosol dose-metering valve, the patient inhales the drug particles into the lungs or nasal cavity. Systems of dispensing drugs in this way are known as “pressurised metered dose inhalers” (pMDIs). See Peter Byron, Respiratory Drug Delivery, CRC Press, Boca Raton, Fla. (1990) for a general background on this form of therapy.

Patients often rely on medication delivered by pMDIs for rapid treatment of respiratory disorders, which are debilitating and in some cases even life threatening. Therefore, it is essential that the prescribed dose of aerosol medication delivered to the patient consistently meets the specifications claimed by the manufacturer and meets the requirements of regulatory authorities. That is, every dose in the can must be delivered within the same close tolerances.

A problem which can exist with drug delivery devices such as pMDIs is deposition of medicament, or the solid component from a suspension of a particulate product in a liquid propellant, onto the internal surfaces of the device. A reduction in the efficacy of the device may occur. Deposition of the product also reduces the amount of active drug available to be dispensed to the patient and markedly reduces the uniformity of the doses dispensed during the lifetime of the device.

Drug deposition and adherence, with consequent loss in dose uniformity, may be greater with formulations comprising hydrofluoroalkane propellants, for example, 1, 1,1,2-tetrafluoroethane (HFA-134a) and 1,1,1,2,3,3,3-n-heptafluoropropane (HFA-227), which have been developed as ozone friendly replacements of chlorofluorocarbons such as P11, P114 and P12.

Some conventional devices rely on the dispenser being shaken, to agitate the liquid propellant and product mixture therein, in an attempt to re-suspend at least a portion of the deposited medicament. While in some cases this remedy can be effective within the body of the drug container itself, it may not be effective for particles deposited on the inner surface(s) of other pMDI components, such as the metering valve.

Canadian patent application 2130867 describes a pMDI having a pressurised metal container which contains an aerosol formulation and which has internal walls coated with a cross-linked plastics coating. Polytetrafluoroethylene (PTFE) and perfluoroethylenepropylene (FEP) are specifically mentioned as suitable coating materials

UK patent application GB-A-2,328,932 discloses the use of a liner of a material such as fluoropolymer, ceramic or glass to line a portion of the wall of the metering chamber in a metering valve of a pMDI. Although this alleviates the problem of deposition in these types of dispensers, it does require the re-design or modification of mouldings and mould tools for producing the valve members to allow for insertion of the liner.

European patent No. 1066073 makes known preventing adhesion of a medicament on internal surfaces of a metering valve of a pMDI by depositing on the internal surfaces a layer of a cold plasma polymerised fluorinated hydrocarbon.

It is an aim of the present invention to provide a medicament dispenser with a medicament-contacting surface which prevents or inhibits adhesion of the medicament thereto.

### SUMMARY OF THE INVENTION

According to the present invention there is provided a component of a medicament dispenser having a surface which, in use of the dispenser, contacts a medicinal formulation contained in the dispenser, said surface being presented by a fluorinated polymeric composition having CF₃CHFCF₃ as a monomer thereof.

For the avoidance of doubt, the term “having CF₃CHFCF₃ as a monomer thereof” encompasses the case where the fluorinated polymeric composition contains CF₃CHFCF₃ as a repeating unit, but also the case where CF₃CHFCF₃ is simply a monomer used in the polymerisation process forming the composition, since some polymerisation processes may result in the monomer changing its chemical structure in the process. This may, for example, occur where the fluorinated polymeric composition is a plasma polymer, that is to say, produced by plasma polymerisation of CF₃CHFCF₃, e.g. cold plasma polymerisation.

CF₃CHFCF₃ is the chemical formula of HFA-227 supra. Thus, the present invention has particular, but not exclusive, utility in alleviating the problem of adherence to the medicament component of an aerosol formulation having HFA-227 as the propellant.

Accordingly, the component is preferably a component of a medicinal aerosol dispenser adapted for dispensing a medicinal aerosol formulation, e.g. the container or one of more of the parts of the valve assembly.

Suitably, the valve component is made of a non-metal material, such as pharmacologically resilient polymers such as acetal, polyamide (e.g. Nylon®), polycarbonate, polyester (e.g. polybutylene terephthalate (PBT)), fluorocarbon polymer (e.g. Teflon®) or a combination of these materials. Alternatively, the valve component is made of metal, for example stainless steel, aluminium, copper, tin plate and any alloys thereof.

The container is typically made of a metal, for instance aluminium or an alloy thereof. However, other metals not affected by the drug formulation, such as stainless steel, an alloy of copper, or tin plate, may be used. The container may also be fabricated from glass or plastics. The container, when for in use in an aerosol dispenser, is a pressurised container.

Other preferred features and aspects of the present invention are set forth in the appended claims and the following detailed description of embodiments of the invention.

### DETAILED DESCRIPTION OF EMBODIMENTS OF THE INVENTION

In accordance with the invention, a batch of polybutylene terephthalate (PBT) metering chambers for a metering valve assembly for a PMDI have been coated on all their surfaces with a fluorinated polymeric composition according to the present invention by cold plasma polymerisation, as further detailed hereinafter.

The valve assembly is of the form shown and described in European patent No. 1066073, the entire content of which is hereby incorporated herein by reference. Thus, the metering chambers are each in the form of a cylindrical sleeve which, in the final valve assembly, is sealed at its opposed, open ends by annular, elastomeric seals and a valve stem which sealingly slides through the seals. The sealed inner volume bounded by the inner surfaces of the sleeve, the outer surface of the valve stem and the seals defines the metered volume of the aerosol formulation dispensed by the valve assembly from a pressurised canister to which it is secured.

The fluorinated coating of the invention is prepared using a RF cold plasma polymerisation process operating at a frequency of about 13.56 MHz. Firstly, the metering chambers are placed inside a rotating reactor chamber so that they are positioned within the primary plasma in the reactor (inside the glow of the plasma). The reactor chamber is then evacuated and operated to rotate at a tumbler speed in the range of about 3 rpm to about 8 rpm. At this stage, the CF₃CHFCF₃ monomer is introduced into the chamber in gaseous form, at ambient temperature and at a controlled flow rate in the range of about 75 cc/min to about 100 cc/min. The monomer gas is then ignited and dissociates into plasma within the reactor chamber. The reactor chamber is controlled to operate at a gas pressure of less than or equal to about 70 mTorr, and at a power of about 200 W.

During the plasma polymerisation, the electrode temperatures increase from about 20° C. to about 100° C. A cooling system of the electrode is used to minimise the temperature increase.

At the end of the treatment the plasma is extinguished, the chamber flushed with air or argon and the coated metering chambers retrieved.

The polymerisation process is carried out for a time which results in a thin layer of fluorinated polymer of no more than about 200 nm being bonded to the surfaces (external and internal) of the metering chambers.

To improve adhesion of the fluorinated coating to the metering chamber surfaces, the surfaces may be subjected to a pre-treatment procedure to remove any surface contamination and/or to activate the surface. The pre-treatment step may be carried out by plasma treatment of the chambers with an etching gas such as oxygen or a neutral gas such as argon. Preferably, the gas is argon to avoid damage to the chamber surfaces. In the process, radicals react with the chamber surfaces; for example exposing the chamber surfaces to a low pressure argon plasma environment generates polar groups on the chamber surfaces. Such polar groups are more conducive to bonding with the fluorine-containing plasma coating of the invention.

The pre-treatment step, for example with argon, could be carried out under a range of conditions and duration. However, the following conditions provide a satisfactory pre-treatment for a PBT metering chamber: run time 5 minutes; power 300 W; gas pressure 80 mTorr; gas flow 150 cc/min; tumbler speed 3 rpm or 8 rpm.

As will be understood by the skilled addressee in the art, other components of a pMDI, such as the canister, or other parts of the valve assembly, may be coated in this way on the surfaces thereof which contact the pharmaceutical aerosol formulation, thereby reducing or eliminating the tendency for medicament particles to adhere to such component surfaces, especially when the pMDI is to be used with an aerosol formulation having HFA-227 as the propellant thereof.

As known by a person skilled in the art, the valve assembly suitably comprises a number of components or parts. Component parts of the valve assembly which may be coated include, but are not limited to, the valve body, sampling chamber, valve stem, the upper and lower stem seals, neck gasket, spring, body, and the ring.

In an alternative embodiment of the invention, the fluorinated CF₃CHFCF₃ monomer may be co-polymerised with one or more additional non-fluorinated monomers. In general the preference is to use a non-fluorinated monomer that forms the basic building block (monomer) of the substrate polymer or elastomer to be coated. For example, if polybutylene terephthalate (PBT) is the substrate to be coated, the monomer used in producing PBT, dimethyl terephthalate, can be used in conjunction with the fluorinated monomer. Similarly, if the substrate is acetal, then CH₂O can be used. Irrespective of the substrate material, it may be desirable to use basic hydrocarbon monomers, including, but not limited to, CH₄, C₂H₆, C₂H₄, N₂, O₂, H₂, C₃COO(C₆H₆)COOCH₃, HO(CH₂)₂OH, C₃H₃N and C₄H₆ in conjunction with the fluorinated monomer.

The ratio of the gas flow rate of the fluorinated monomer gas to the non-fluorinated monomer gas can be continuously varied during the course of the plasma coating process. In general, in order to obtain superior adhesion, this ratio can be low or, expressed another way, the monomer gas can be rich in the non-fluorinated species at the start of the process. This ratio can be continuously increased and towards the end of the process it is preferable to use only the fluorinated monomer in order to obtain a fluorine rich surface in the top layers of the coating.

Of course, the ratio of the gas flow rates of the monomers can be maintained constant instead.

Medicaments which may be administered as aerosol formulations include drugs useful in inhalation therapy. The dispenser of the invention may be used for dispensing medicament for the treatment of respiratory disorders such as disorders of the lungs and bronchial tracts including asthma and chronic obstructive pulmonary disorder (COPD). The dispenser of the invention may also be used for dispensing medicament for the treatment of a condition requiring treatment by the systemic circulation of medicament, for example migraine, diabetes, pain relief e.g. inhaled morphine.

Appropriate medicaments may be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate or furoate ester), flunisolide, budesonide, rofleponide, mometasone (e.g. as the furoate ester), ciclesonide, triamcinolone (e.g. as the acetonide) or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino]propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g. hydrates) to optimise the activity and/or stability of the medicament.

Preferred medicaments are selected from albuterol, salbutamol, salmeterol, fluticasone propionate and beclomethasone dipropionate and salts or solvates thereof, e.g., the sulphate of albuterol and the xinafoate of salmeterol.

Medicaments can also be delivered in combinations. Preferred formulations containing combinations of active ingredients contain salbutamol (e.g., as the free base or the sulphate salt) or salmeterol (e.g., as the xinafoate salt) or formoterol (e.g. as the fumarate salt) in combination with an anti-inflammatory steroid such as a beclomethasone ester (e.g., the dipropionate) or a fluticasone ester (e.g., the propionate) or budesonide. A particularly preferred combination is a combination of fluticasone propionate and salmeterol, or a salt thereof (particularly the xinafoate salt). A further combination of particular interest is budesonide and formoterol (e.g. as the fumarate salt).

Particularly preferred aerosol formulations for use in the dispenser of the present invention comprise a medicament and a propellant consisting of, or including, 1,1,1,2,3,3,3-n-heptafluoropropane (HFA-227).

Administration of the medicament may be indicated for the treatment of mild, moderate or severe acute or chronic symptoms or for prophylactic treatment.

It will be appreciated that the exemplary embodiments described above are by way of illustration of the invention, not limitation, and that the invention can take on numerous other guises and forms within the scope of the appended claims.

For the avoidance of doubt, the use herein of terms such as “about”, “substantially” and the like in relation to the value(s) of certain parameters is meant to include the exact value of that parameter as well as minor, inconsequential deviations therefrom.

What is claimed is:

1. A component of a medicament dispenser having a surface which, in use of the dispenser, contacts a medicinal formulation contained in the dispenser, said surface being presented by a fluorinated polymeric composition having CF₃CHFCF₃ as a monomer thereof.

2. The component of claim 1 which is a component of a medicinal aerosol dispenser adapted for dispensing a medicinal aerosol formulation.

3. The component of claim 1 which is a container with the surface being an interior surface of the container which bounds an inner volume for receiving the medicinal formulation.

4. The component of claim 3 in which the interior surface consists of all, or substantially all, of the interior surfaces of the container which bound the inner volume.

5. The component of claim 1 which is a component of a valve assembly adapted to operate to dispense the formulation from the dispenser.

6. The component of claim 5 wherein the valve assembly is a metering valve which operates to dispense metered volumes of the medicinal formulation from the dispenser.

7. The component of claim 5 which is a chamber of the valve assembly, the surface being an interior surface of the chamber which bounds an inner volume which, in use, stores a volume of the formulation.

8. The component of claim 7, wherein the chamber is a metering chamber, the inner volume operating to dose a metered volume of the formulation.

9. The component of claim 7, wherein the interior surface consists of all, or substantially all, of the interior surfaces of the chamber which bound the inner volume.

10. The component of claim 5 which is an outlet member of the valve assembly having an internal passageway through which the formulation is dispensed by the valve assembly.

11. The component of claim 10, wherein the surface is an external surface of the outlet member.

12. The component of claim 10, wherein the surface bounds the internal passageway.

13. The component of claim 1 wherein the polymeric composition is formed exclusively, or substantially exclusively, of or from CF₃CHFCF₃ monomer.

14. The component of claim 1 wherein the polymeric composition is formed by polymerisation of CF₃CHFCF₃ with one or more other monomers.

15. The component of claim 14 in which the other monomer, or at least one of the other monomers, is a fluorinated monomer.

16. The component of claim 14 in which the other monomer, or at least one of the other monomers, is a non-fluorinated monomer.

17. The component of claim 16, wherein the non-fluorinated monomer is selected from the group consisting of CH₄, C₂H₆, C₂H₄, N₂, O₂, H₂, C₃COO(C₆H₆)COOCH₃, HO(CH₂)₂OH, C₃H₃N and C₄H₆.

18. The component of claim 1 wherein the polymeric composition is in the form of a coating deposited on the component surface.

19. The component of claim 18, wherein the coating has a thickness in the range of about 1 nm to about 200 nm.

20. The component of claim 18, wherein the coating is on a metal, a plastic or a ceramic undersurface of the component.

21. The component of claim 20 which is a metal, plastic or ceramic component.

22. The component of claim 1 in which the polymeric composition is formed by plasma polymerisation.

23. A medicament dispenser comprising the component of claim 1.

24. The medicament dispenser of claim 23 in the form of an aerosol dispenser.

25. The medicament dispenser of claim 23 having the container with the surface being an interior surface of the container which bounds an inner volume for receiving the medicinal formulation.

26. The medicament dispenser of claim 23 having a valve assembly comprising at least one of the components.

27. The medicament dispenser of claim 23 containing a medicinal aerosol formulation which includes HFA-227 as a propellant.

28. The medicament dispenser of claim 23 forming a part of a pressurised, metered dose inhaler (pMDI).

29. A valve assembly for a medicament dispenser having the component of claim 1.

30. A component of a medicament dispenser having a surface which, in use of the dispenser, contacts a medicinal formulation contained in the dispenser, said surface being presented by a fluorinated polymeric composition formed by a polymerisation process using CF₃CHFCF₃ as a monomer.

31. The component of claim 30, wherein the polymerisation process is plasma polymerisation.

32. The component of claim 31, wherein the polymerisation process is cold plasma polymerisation.

33. (canceled)
